(19) United States
(12) Patent Application Publication
(10) Pub. No.: US 2025/0295331 A1
(43) Pub. Date: Sep. 25, 2025

(54) METHOD FOR MEASURING RELATIVE MOVEMENT BETWEEN BONES AND METHOD FOR ACQUIRING JOINT ROTATION AXIS OF BONES

(71) Applicant: THE SECOND XIANGYA HOSPITAL OF CENTRAL SOUTH UNIVERSITY, Changsha (CN)

(72) Inventor: Liwen ZHENG, Changsha (CN)

(73) Assignee: THE SECOND XIANGYA HOSPITAL OF CENTRAL SOUTH UNIVERSITY, Changsha (CN)

(21) Appl. No.: 19/074,546

(22) Filed: Mar. 10, 2025

Related U.S. Application Data
(63) Continuation-in-part of application No. PCT/CN2023/117233, filed on Sep. 6, 2023.

(30) Foreign Application Priority Data
Sep. 10, 2022 (CN) 202211106267.X

(51) Int. Cl. A61B 5/11; A61B 5/00

(52) U.S. Cl. CPC A61B 5/1121; A61B 5/0033; A61B 5/1128; A61B 5/4528

(57) ABSTRACT

A method for measuring a relative movement between bones and a method for acquiring a joint rotation axis of bones are provided, which can effectively improve rotational stability of a prosthesis of knee arthroplasty, thereby improving surgical outcomes. A population average bone joint most stable axis (a-MSA) is acquired through the method, so that the design of a joint prosthesis can be improved and a reference of a population average position can be provided for intraoperative placement of a prosthesis. An individual bone joint MSA (i-MSA) of a patient is acquired through the method before surgery, so that individual position information can be provided for intraoperative placement of a prosthesis, thereby further improving rotational stability of a surgical prosthesis.

### CROSS REFERENCE TO THE RELATED APPLICATIONS

This application is a continuation-in-part application of International Application No. PCT/CN2023/117233, filed on Sep. 6, 2023, which is based upon and claims priority to Chinese Patent Application No. 202211106267.X, filed on Sep. 10, 2022, the entire contents of which are incorporated herein by reference.

### TECHNICAL FIELD

The present invention relates to the fields of biomechanics and surgery, and in particular, to a method for measuring a relative movement between bones and a method for acquiring a joint rotation axis of bones.

### BACKGROUND

Total knee arthroplasty (TKA) is an orthopedic surgery performed in very large numbers currently and is mainly used to treat patients with end-stage knee osteoarthritis (OA). However, according to surveys, 8% to 25% of patients are dissatisfied with surgical outcomes currently. To improve surgical outcomes, researchers have conducted many studies in the last two decades, and a knee joint rotation axis is considered to be an important factor affecting surgical outcomes. The design of TKA artificial joints is based on acquisition of an accurate population average rotation axis, and intraoperative placement of a joint prosthesis is based on acquisition of an accurate individual rotation axis of a patient.

To localize this axis, a knee joint rotation axis needs to be clearly defined first. However, if a conventional definition of a rotation axis is directly applied to an application scenario of a knee joint, the following problem arises: The definition of a rotation axis is clear for a strictly pivotal movement because there is an absolutely stable rotation axis for the strictly pivotal movement. However, a knee joint movement is mainly a pivotal movement (flexion-extension), and is also combined with various movements including internal-external rotations and varus-valgus rotations. These movements are subject to individual variations. In other words, the knee joint movement is complex, and there is no absolutely stable rotation axis. Therefore, the conventional definition of the rotation axis cannot be directly applied to a knee joint. Currently, many papers focus on a knee joint rotation axis, but no researcher has clearly described a precise definition of a knee joint rotation axis, leading to the problem that the design and placement of an artificial joint fail to accurately adapt to individuals during surgery.

### SUMMARY

To solve the technical problem that currently a joint rotation axis cannot be accurately localized, leading to suboptimal surgical outcomes of arthroplasty, the present invention provides a method for acquiring a joint rotation axis of bones.

To achieve the foregoing technical objective, according to a first aspect, the present invention provides a method for measuring a relative movement between bones, including the following steps:
S11: imaging, by a magnetic resonance imaging (MRI) device, bones before a movement for the first time to obtain a first image;
S12: imaging, by the MRI device, the bones after the movement for the second time to obtain a second image, and then rotating the second image to make an orientation of the imaging of the bones in the second image same as that in the first image, where a reference for determining whether the orientations are the same is to compare characteristic area frameworks (CAFs) to determine whether the CAFs are consistent, each of the CAFs is formed based on a plurality of characteristic areas in the bones, and the characteristic areas are subcortical vessels (SCVS) of the bones; and
S13: measuring, by the MRI device, a difference between angles of the second image before the rotation and after the rotation and a displacement distance between the second image after the rotation and the first image, and calculating a value of a relative movement between the bones based on the difference between the angles and the displacement distance.

As a further improvement to the foregoing solution, in step 12, the characteristic areas are areas in which vessels lie immediately beneath bone cortices and traverse the bone cortices.

As a further improvement to the foregoing solution, in step 12, the CAF includes at least two characteristic areas in different slices in an MRI image, and an interval between the characteristic areas is not less than a preset quantity of MRI slices; and a size of each of the characteristic areas does not exceed a preset size limit.

According to a second aspect, the present invention further provides a method for acquiring an individual bone joint most stable axis (i-MSA), including the following steps:
S1: acquiring values of a 6-degree-of-freedom (DOF) relative movement between bones of an individual target joint at different bending angles based on the method for measuring a relative movement between bones provided in the first aspect or a conventional measurement method;
S2: performing three-dimensional (3D) meshing with a preset mesh size on the bones of the individual target joint through a computer-aided tool, and randomly selecting one point in each mesh to form a candidate point set;
S3: quantitatively assessing stability of each point in the candidate point set in step S2 using the values of the relative movement obtained in step S1, and selecting a preset quantity of most stable points to form a most stable point group; and
S4: finding one straight line closest to all points in the most stable point group using a least squares method or gradient descent method as an i-MSA of the individual target joint.

As a further improvement to the foregoing solution, in step S3, a method for assessing the stability of the point is as follows:
using a point with stability to be assessed as a target point, and obtaining, according to the values of the relative movement between the bones at the different angles of the joint, a position coordinate of the target point at each of the angles; and then calculating an average coordinate of these position coordinates, measuring a distance of the average coordinate from each of the position coordinates, and then calculating a mean square error or an arithmetic average value of these distances, where when the mean square error or arithmetic average value is larger, stability of the target point is poorer, and when the mean square error or arithmetic average value is smaller, the stability of the target point is better.

As a further improvement to the foregoing solution, in step S3, a method for acquiring the preset quantity of the points in the most stable point group is as follows:
when a unified mesh obtained by splicing all meshes in step S2 is a standard body, selecting a preset proportion of points with the best stability as the most stable point group, where the standard body is the smallest cube that can accommodate an entire bone at a distal end of a bone on a side of the joint close to a human head; and when a volume of the unified mesh is different from that of the standard body, adjusting, according to an inverse proportion of the volumes of the unified mesh and the standard body, the preset proportion for selecting points.

According to a third aspect, the present invention further provides a method for acquiring a population average MSA (a-MSA) applicable to knee joints and elbow joints, including the following steps:
S1: acquiring transepicondylar axes (TEAs) of joints under test of a plurality of targets through a computer-aided measurement tool;
S2: acquiring individual most stable axes of all the joints under test in step S1 based on the method for acquiring an individual MSA provided in the second aspect;
S3: determining, through the computer-aided measurement tool, a 3D space coordinate system in which a joint is located: using a straight line in which a TEA is located as an X axis; and then drawing an inscribed circle of a diaphyseal medullary cavity of a femur/humerus at a transverse height of a standard length on a proximal side of the TEA, and drawing a perpendicular line from a center of circle to the X axis as a Z axis, where a direction of a Y axis is perpendicular to both the X axis and the Z axis;
S4: determining a medial sagittal plane coordinate system and a lateral sagittal plane coordinate system through the computer-aided measurement tool: using a plane that is perpendicular to the X axis and passes through a medial epicondyle of the femur/humerus as a medial sagittal plane, and using a plane that is perpendicular to the X axis and passes through a lateral epicondyle of the femur/humerus as a lateral sagittal plane; on the medial sagittal plane, establishing a two-dimensional (2D) coordinate system, referred to as the medial sagittal plane coordinate system, with the medial epicondyle of the femur/humerus as an origin and the Y axis and the Z axis as coordinate axis directions; and on the lateral sagittal plane, establishing a 2D coordinate system, referred to as the lateral sagittal plane coordinate system, with the lateral epicondyle of the femur/humerus as an origin and the Y axis and the Z axis as coordinate axis directions; and
S5: for an intersection between the individual MSA of each joint under test and the medial sagittal plane, representing an anterior-posterior position, i.e., a Y-axis coordinate, of the intersection in the medial sagittal plane coordinate system using a parameter medial-anterior-posterior (M-AP); representing a proximal-distal position, i.e., a Z-axis coordinate, of the intersection using a parameter medial-proximal-distal (M-PD); representing an anterior-posterior position, i.e., a position on the Y axis, of an intersection between the individual MSA and the lateral sagittal plane using a parameter lateral-anterior-posterior (L-AP); representing a proximal-distal position, i.e., a position on the Z axis, of the intersection using a parameter lateral-proximal-distal (L-PD); and taking population average values of the four parameters, i.e., a population average relative position relationship between the individual MSA and the TEA, to acquire an a-MSA.

As a further improvement to the foregoing solution, in step S3, the standard length is a distance between a medial epicondyle and a lateral epicondyle of the TEA×a, where a is a coefficient and has a value ranging from 0.44 to 1.0.

The technical effect of the present invention lies in that the present invention can effectively improve rotational stability of a prosthesis of knee arthroplasty, thereby improving surgical outcomes. An a-MSA is acquired through the method provided in the present invention, so that the design of a joint prosthesis can be improved and a reference of a population average position can be provided for intraoperative placement of a prosthesis. An i-MSA of a patient is acquired through the method provided in the present invention before surgery, so that individual position information can be provided for intraoperative placement of a prosthesis, thereby further improving rotational stability of a surgical prosthesis.

### BRIEF DESCRIPTION OF THE DRAWINGS

FIGS. 1A-1F are schematic diagrams describing a method for standardizing a spatial position of a rotation axis near a knee joint according to the present invention, where FIG. 1A to FIG. 1D are schematic diagrams of determining 3D directions of a medial sagittal plane and a lateral sagittal plane based on a distal anatomical structure of a femur, and FIG. 1E and FIG. 1F are schematic diagrams of establishing 2D coordinate systems on the medial sagittal plane and the lateral sagittal plane for describing the position of the rotation axis; in the figures, a TEA is an X axis, a Z axis is a line connecting a midpoint of the TEA and the center of circle of an inscribed circle of a distal medullary cavity of the femur, and a Y axis is perpendicular to the X axis and the Z axis; and sagittal planes in which a medial end and a lateral end of the TEA are located are respectively the medial sagittal plane and the lateral sagittal plane;

FIGS. 2A-2E are schematic diagrams of the principle of a method for measuring a relative movement between bones disclosed in the present invention, where FIG. 2A shows an initial movement state of a rigid body; FIG. 2B shows a final movement state of the rigid body, and FIG. 2C and FIG. 2E show the principle of measuring a relative movement of a 2D rigid body; FIG. 2C, FIG. 2D, and FIG. 2E show the principle of measuring a 3D rigid body; i.e., a process from FIG. 2C straight to FIG. 2E is used to a measure a relative movement of a 2D rigid body; a process from FIG. 2C to FIG. 2D to FIG. 2E is used to measure a relative movement of a 3D rigid body; i.e., during the measurement of the relative movement of the 3D rigid body, FIG. 2D, i.e., the rotation of the 3D rigid body, is required before measurement; for the 2D rigid body, there is no process of rotation, i.e., the process in FIG. 2D, is not required; and M in the figures is a moving rigid body, F is a fixed rigid body, P and P′ are respectively points before and after the moving rigid body has moved, V and V′ represent a displacement vector of the point, and θ is a rotation angle of the moving rigid body M;

FIGS. 3A-3L show forward and reverse directions of rotations of a coronal plane, a sagittal plane, and a transverse plane and a CAF formed by a plurality of SCVS, where FIG. 3A to FIG. 3D show the coronal plane, FIG. 3E to FIG. 3H show the sagittal plane, and FIG. 3I to FIG. 3L show the transverse plane; and small circles in the figures are SCVS selected as characteristic areas, T is a tibia, P is a patella, Fm is a femur, and Fb is a fibula;

FIGS. 4A-4F are schematic diagrams of changes in SCVS after a movement, where FIG. 4A shows an image of a coronal plane in an MRI of a knee joint before a rotation/translation, the SCVS are marked with a small circle, FIG. 4B shows an image of a coronal plane after FIG. 4A is rotated by 0.5° in a sagittal plane, and at this time the SCVS has changed; FIG. 4C shows an image obtained by displacing FIG. 4A by 0.5 mm perpendicular to the coronal plane, and at this time the SCVS has also changed; FIG. 4D to FIG. 4F are similar to FIG. 4A to FIG. 4C, except that a tibia and a femur are replaced with aquarium rocks; and the rotation of 0.5° or the displacement of 0.5 mm causes significant changes in feature areas; and

FIGS. 5A-5E are schematic diagrams of the arrangement of a mesh body, where FIG. 5A to FIG. 5C are schematic diagrams of a cubic mesh with side lengths being an interepicondylar width at a distal end of a femur, with a mesh spacing of 1 mm and all mesh intersections being candidate points; and the white asterisks in FIG. 5D and FIG. 5E represent candidate points in the top 0.2% with the smallest positional change (PC) values for determining an i-MSA.

### DETAILED DESCRIPTION OF THE EMBODIMENTS

The concepts of directions (medial, lateral, proximal, distal, anterior, and posterior) used throughout the present invention are common knowledge in the related professions. The present invention describes joint directions in anatomical positions, a medial/lateral side is a side close to/far away from the longitudinal center axis of the human body, and a proximal/distal side is a side close to the head/feet.

First, the present invention no longer pursues absolute stability in finding a knee rotation axis, but instead designs a quantitative indicator for the degree of stability, so that: (1) degrees of stability of different rotation axes can be compared based on the indicator, and (2) the MSA can be found according to the indicator. Theoretically, when a prosthesis in knee arthroplasty (including TKA and unicompartmental knee arthroplasty (UKA)) has a more stable rotation axis, the movements of a postoperative knee joint are closer to those of a natural knee joint, a patient feels less discomfort, and surgical outcomes are better.

To implement the assessment of the stability of a knee joint rotation axis, the following steps are logically required: (1) A value of a relative movement between bones on two sides of a joint when the joint is bent at different angles is measured. (2) The bone on one side of the joint is used as a fixed bone, the bone on the other side of the joint is used as a moving bone, a position coordinate of a point on the moving bone when the joint is bent at any angle is calculated based on the value of the relative movement, a movement trajectory of the point is obtained, where when position coordinates on the movement trajectory are more dispersed, the stability of the point is poorer, when the position coordinates on the movement trajectory are more concentrated, the stability of the point is better, and a degree of dispersion is represented by a PC quantity (a PC value), and this step is repeated to obtain a PC value of each point on the moving bone. (3) If all points on one straight line are very stable (have very small PC values), a rotation axis represented by this straight line is also very stable. (4) When two points on a bone are closer to each other, movement trajectories of the two points are more similar, and PC values of the two points are also closer. Therefore, PC values of different points on the rotation axis change continuously. Therefore, points (intersections between the rotation axis and two sagittal planes passing through a medial epicondyle and a lateral epicondyle of a femur) at two standard positions may be taken, and PC values of the points reflect the stability of the entire rotation axis. It can be predicted that the accuracy of the method for assessing stability constructed with the foregoing logic mainly depends on measurement accuracy of a relative movement between bones on two sides of a joint.

Six DOFs are required for a measurement result of a relative movement in academic papers currently. Three of the DOFs are used to describe three rotational directions in a 3D space, and the other three of the DOFs are used to describe three translational directions in the 3D space. Therefore, the assessment of the measurement accuracy also needs to correspondingly include two aspects: translation accuracy and rotation accuracy. At present, the most popular technique is a 2D-3D image matching technique based on X-ray radiography, which has translation accuracy less than 1 mm and rotation accuracy less than 1° [“Accuracy of mobile biplane X-ray imaging in measuring 6-DOF patellofemoral kinematics during overground gait”, Journal of Biomechanics, Vol. 24, No. 57, pp. 152-156, 2017]. Similarly, the accuracy of a computed tomography (CT) measurement technique is also not inferior to that of an X-ray radiography technique [“Implant placement accuracy in total knee arthroplasty: validation of a CT-based measurement technique”, Quant Imaging Med Surg, Vol. 2, No. 10, pp. 475-484, 2020]. Both the techniques have very good accuracy, but have limited clinical applications due to high radiation doses, and are primarily used for scientific research. The MRI technique has no radiation, but the measurement accuracy in the literature only ranges from 3 mm to 7 mm and 3° to 4° [Development and Validation of a Subject-specific Moving-axis Tibiofemoral Joint Model Using MRI and EOS Imaging during a Quasi-Static Lunge, Journal of Biomechanics, Vol. 27, No. 72, pp. 71-80, 2018]. In the present invention, SCVS form a CAF to improve measurement accuracy of an MRI to be less than 1 mm and less than 1°, thereby providing a non-radioactive measurement method.

The main conventional concepts of knee joint rotation axes in the current literature include a TEA, a geometric center axis (GCA), a posterior condylar axis (PCA), and a Whiteside's line (WSL). According to the definitions, the knee joint rotation axes may include 2D rotation axes (the PCA and the WSL) and 3D rotation axes (the GCA and the TEA). However, the definition of a 2D rotation axis is insufficient. A position of the rotation axis in a coronal plane is not defined, and in a transverse plane, only a direction of the rotation axis is defined but an anterior-posterior position is not defined. As a result, a spatial position of the rotation axis cannot be determined, making it impossible to assess the stability of the rotation axis. The 3D rotation axes are well defined, so that the stability of the 3D rotation axes is measured in the present invention, and the 3D rotation axes are compared with rotation axes i-MSA and a-MSA proposed in the present invention.

The content of the present invention is described below through specific embodiments.

### 1. Acquisition of a Value of a Relative Movement Between Bones

The three imaging techniques, namely, X-ray radiography, CT, and MRI, can all be used to measure a relative movement between bones, to obtain a value of a 6-DOF relative movement of bones. The measurement techniques of X-ray radiography and CT have long existed and have been extensively applied to the scientific research field. Details are not described herein. An MRI measurement method (referred to as a novel MRI measurement method below) used in this embodiment is mainly described herein.

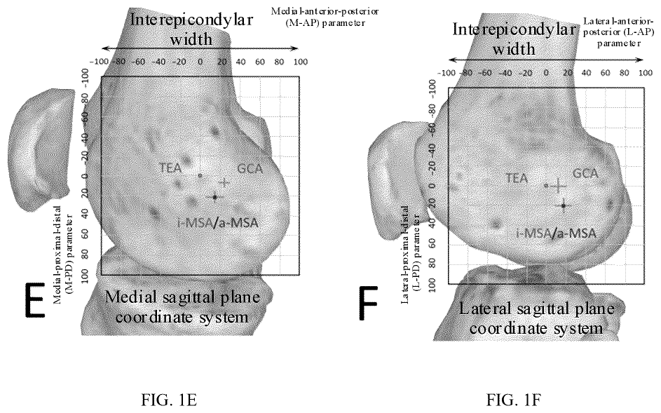

To aid in understanding the principle of the novel MRI measurement method, the case of a 2D rigid body is examined first. Referring to FIGS. 2A-2E, a relative movement between two rigid bodies may be described through two parameters: (1) a rotation angle θ of a rigid body; and (2) a displacement V of any point P on the rigid body. Because the rotation angle θ of the 2D rigid body and the displacement V may be directly measured on an image, it can be relatively convenient to obtain a relative movement between the two rigid bodies. After the measurement of θ and Vis completed, a displacement V′ of any other point P′ on the rigid body may be calculated through Formula (1).

$${V}^{′} = ( {P}^{′} - P ) [ \begin{matrix}cos θ , & - sin θ \\ sin θ , & cos θ\end{matrix} ] + V . \qquad ( 1 )$$

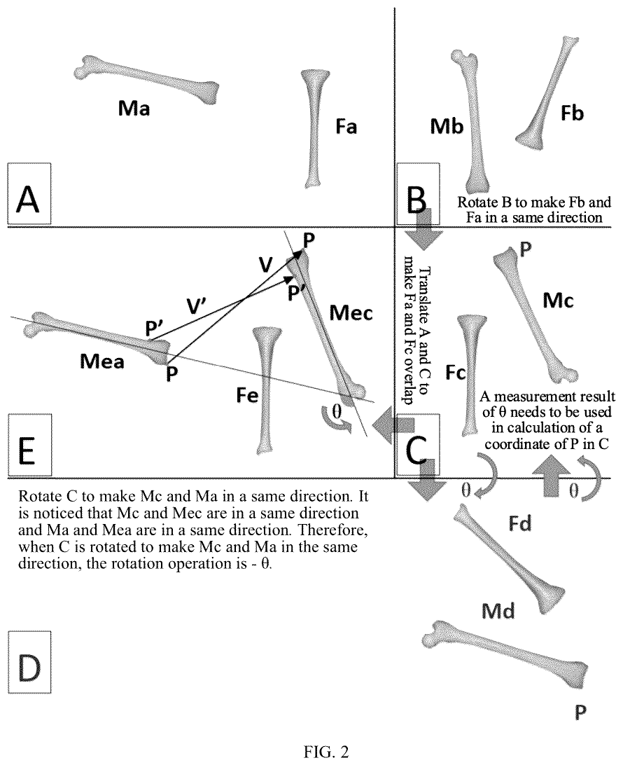

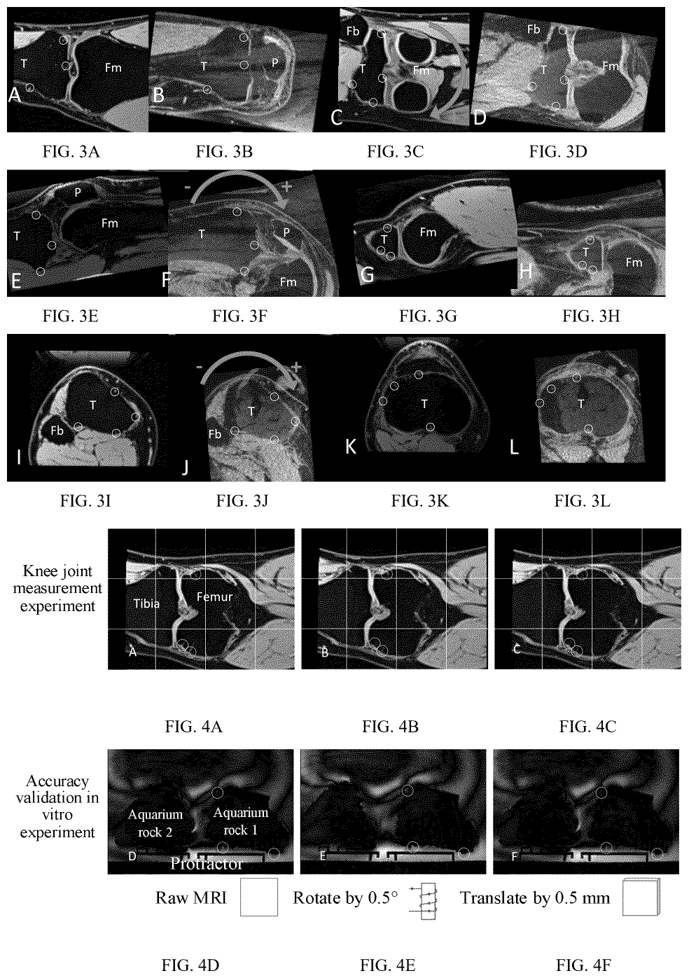

In this case, for a 3D rigid body like a bone, because the complete view of the rigid body cannot be obtained in an MRI image, θ and I cannot be directly measured as in the case of a 2D rigid body. Therefore, in this embodiment, an MRI image of bones after a movement is rotated to make orientations of the bones in images before and after the movement the same, in other words, move the bones in the two images to a same position to overlap. In this case, a rotation angle of the image is equivalent to θ in the case of a 2D rigid body. Certainly, the measurement accuracy of the method depends on how to determine as accurately as possible that the orientations of the bones before the rotation and after the rotation are identical. Referring to FIGS. 3A-3L and FIGS. 4A-4F, in this embodiment, a plurality of SCVS form a CAF for use as a tool for determining alignment accuracy of a 3D rotation. After θ is determined, V may be measured through some simple operations, because after θ is rotated, a translation distance of a same point in two images is Vin a 2D rigid body. After the measurement of θ and V is completed, a displacement V′ of any other point P′ on the rigid body may be calculated through Formula (2).

$${V}^{′} = ( {P}^{′} - P ) RxRyRz + V , \\ where \\ Rx = [ \begin{matrix}1 & 0 & 0 \\ 0 & cos a & - s in a \\ 0 & sin a & cos a\end{matrix} ] , \\ Ry = [ \begin{matrix}cos b & 0 & sin b \\ 0 & 1 & 0 \\ - s in b & 0 & cos b\end{matrix} ] , \\ Rz = [ \begin{matrix}cos c & - sin c & 0 \\ sin c & cos c & 0 \\ 0 & 0 & 1\end{matrix} ] , and \\ θ = [ a , b , c ] . \qquad ( 2 )$$

Because the CAF has a significant impact on measurement accuracy, in this embodiment, a unified standard is adopted for the CAF: (1) The length/width of each characteristic area (SCVS) is less than or equal to 5 pixels (2.5 mm). (2) In each coordinate axis direction, the characteristic areas need to be distributed in two different MRI slices that are more than 80 slices (4 cm) apart. Referring to FIGS. 4A-4F, under the standard, the CAF is sensitive to a minor rotation of a rigid body. A rotation of 0.5° of the rigid body can cause a significant change visible to naked eyes in the CAF. As FIG. 4A to FIG. 4C indicate, provided that one single characteristic area (SCVS) is different, it can be determined that the rigid body has either undergone a slight rotation or a slight translation. Therefore, if the same CAF is found in MRI images of two rigid bodies, a directional difference between the two rigid bodies is less than 0.5°. Therefore, relying on the CAF, in conjunction with the description of the principle section, a rotation parameter θ of a rigid body can be accurately measured. Similarly, the CAF is also sensitive to a minor translation of a rigid body (a translation of 0.5 mm can cause a significant change visible to naked eyes in the CAF). Therefore, a translational parameter V of the rigid body can also be accurately measured.

The supplementary description for the foregoing standard for the CAF is as follows: According to experience, generally, when a characteristic area is smaller, the localization accuracy of a translation is higher, but manual comparison is more difficult. When a CAF is larger, the rotation accuracy is higher, but it is more difficult to find SCVS that meet the standard. The standard for the CAF used in this embodiment is a comprehensive optimal choice according to experience, but close measurement accuracy can also be reached by modifying a size parameter of a characteristic area and a size parameter of a CAF in the standard.

Specifically, referring to FIGS. 2A-2E, a method for measuring a relative movement between bones (i.e., the novel MRI measurement method) based on an MRI device provided in this embodiment includes the following steps:

Step S1: An MRI device images bones before a movement for the first time to obtain a first image.

Step S2: The MRI device images the bones after the movement for the second time to obtain a second image, and then rotates the second image to make an orientation of the imaging of the bones in the second image same as that in the first image, where a reference for determining whether the orientations are the same is to compare CAFs to determine whether the CAFs are consistent, each of the CAFs is formed based on a plurality of characteristic areas in the bones, and the characteristic areas are SCVS of the bones. The characteristic areas are areas in which vessels lie immediately beneath bone cortices and traverse the bone cortices. The CAF includes at least two characteristic areas in different slices in an MRI image, and an interval between the characteristic areas is not less than 80 MRI slices. The length/width of each characteristic area is not greater than 5 pixels.

Step S3: The MRI device measures a difference between angles of the second image before the rotation and after the rotation and a displacement distance between the second image after the rotation and the first image, and calculates a value of a relative movement between the bones based on the difference between the angles and the displacement distance.

In this embodiment, the overall rotation accuracy of a rigid body and the translation accuracy of an intersection in a 3D mesh are validated in in vitro measurement experiments, as shown in Table 1. Results show that all examined mesh intersections have translation accuracy less than 1 mm, and the rotation accuracy of a rigid body is less than 1°. Measurement ranges of a rotation of 0° to 150° and a translation of 0 mm to 100 mm are validated in this embodiment. The ranges are sufficient for knee joint measurement.

TABLE 1
Measurement accuracy examination
Measurement accuracy of V and θ

| Actual translation | | | |
|---|---|---|---|
| distance/rotation | Measurement accuracy (a confidence | | |
| angle | interval of 95%)*# | ICC within-rater* | ICC between-rater* |
| Translation | | | |
| (millimeter) | | | |
| 1 | [0.00, 0.00]/[0.00, 0.00]/[0.00, 0.00] | 1.000/1.000/1.000 | 1.000/1.000/1.000 |
| 2 | [0.00, 0.00]/[0.00, 0.00]/[0.00, 0.00] | | |
| 5 | [0.00, 0.00]/[0.00, 0.00]/[0.00, 0.00] | | |
| 10 | [0.00, 0.00]/[−0.79, 0.04]/[−0.68, 0.18] | | |
| 20 | [0.00, 0.00]/[0.00, 0.00]/[−0.09, 0.034] | | |
| 50 | [−0.61, 0.86]/[−0.68, 0.18]/[−0.67, 042] | | |
| 100 | [−0.61, 0.86]/[−0.68, 0.18]/[−0.68, 0.18] | | |
| Rotation angle | | | |
| (degree) | | | |
| 1 | [0.00, 0.00]/[0.00, 0.00]/[0.00, 0.00] | | |
| 5 | [0.00, 0.00]/[0.00, 0.00]/[0.00, 0.00] | | |
| 10 | [−0.81, 0.31]/[0.00, 0.00]/[−0.59, −0.16] | | |
| 20 | [−0.31, 0.81]/[0.15, 0.85]/[−0.29, 0.54] | | |
| 50 | [−0.09, 0.34]/[−1.00, 0.00]/[−0.35, 0.35] | | |
| 80 | [−0.18, 0.68]/[0.00, 1.00]/[−0.18, 0.68] | | |
| 150 | [−0.81, 0.68]/[−0.33, 0.85]/[−0.67, 0.18] | | |

Measurement accuracy of V′

| Actual translation | Measurement accuracy (a confidence | | |
|---|---|---|---|
| distance | interval of 95%)*\hat{ } | ICC within-rater* | ICC between-rater* |
| Translation | | | |
| (millimeter) | | | |
| 1 | [−0.02, 0.01]/[−0.00, 0.02]/[−0.01, 0.01] | 1.000/1.000/1.000 | 1.000/1.000/1.000 |
| 2 | [−0.02, 0.01]/[−0.01, 0.01]/[−0.02, 0.01] | | |
| 5 | [−0.01, 0.02]/[−0.03, 0.01]/[−0.02, −0.01] | | |
| 10 | [−0.01, 0.02]/[−0.78, 0.04]/[−0.68, 0.20] | | |
| 20 | [−0.00, 0.01]/[−0.02, 0.01]/[−0.10, 0.35] | | |
| 50 | [−0.63, 0.86/[−0.67, 0.17]/[−0.69, 0.41] | | |
| 100 | [−0.61, 0.87]/[−0.68, 0.17]/[−0.69, 0.18] | | |

*in the table represents that measurement results in three different directions in space are divided by the symbol “/” to indicate a measurement result in a coronal plane/sagittal plane/transverse plane.
#in the table represents that measurement accuracy is obtained by subtracting an actual value from a measured length/angle. Two raters perform measurement twice separately, and a total of four measurement results are obtained. Four measured values are used to calculate an average value and a standard deviation of measurement accuracy.
\hat{ }in the table represents establishing a cubic mesh with a mesh spacing being 1 cm and a side length being 14 cm at a rigid body under test and a surrounding space, and there are a total of 15³ = 3375 mesh intersections. A center point of the mesh is used as P, and the remaining 3374 mesh intersections are used as P′. P′ has 4 * 3374 = 13496 measurement results, which are used to calculate an average value and a standard deviation of measurement accuracy.

### 2. Method for Assessing Stability of a Knee Joint Rotation Axis

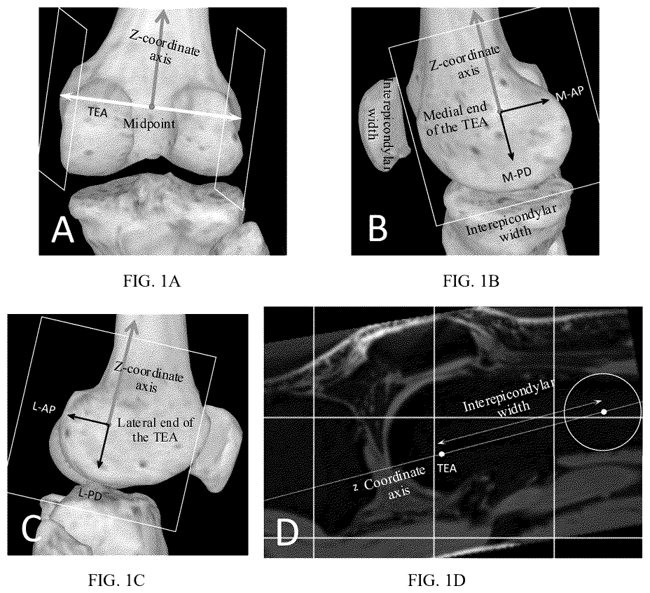

A plurality of MRIs (taken at intervals of 10°, a total of 14 MRIs) with knee joint flexion from 0 to 130° are taken for a single subject. Through the foregoing method, a movement trajectory (the trajectory is formed by 14 coordinates, which respectively represent positions of the point at knee flexion from 0 to 130° of the subject) of any point on a femur with respect to a tibia can be obtained. A mean square error of distances between these coordinates and an average coordinate is used to measure the magnitude (referred to as a PC in this embodiment) of a change in a position of the point in a process of knee flexion. Clearly, if a point has a smaller PC value, a movement trajectory of the point is more compact, and the point is more stable in a movement process of a knee joint. For the rotation axis, referring to FIG. 1E and FIG. 1F, in this embodiment, two sagittal planes on a medial side and a lateral side are first determined, and the rotation axis has two intersections on the medial side and the lateral side with the two sagittal planes. PC values of the two points are used to assess the stability of the rotation axis.

A method for assessing stability of a specific point (a point used as an assessment target, referred to as a target point below) on a bone in this embodiment includes the following steps:

Step 1: For bones on two sides of a joint of a single subject being assessed, acquire MRI images of the joint at different angles.

Step 2: Next, according to the foregoing method for measuring a relative movement between bones, acquire a value of a relative movement between the bones of the joint at the different angles.

Step 3: According to the value of the relative movement between the bones, calculate 14 position coordinates corresponding to a target point at different angles (0 to 130°, at intervals of) 10° of knee flexion, and calculate an average coordinate of the position coordinates. The average coordinate has 14 distances from the 14 coordinates on a movement trajectory, and a mean square error (or an arithmetic average value, a difference in an experimental result being very small) of these distances is calculated as a PC value to assess the stability of the target point.

A method for assessing stability of a specific rotation axis (a rotation axis used as an assessment target, referred to as a target rotation axis below) in this embodiment includes the following steps:

Step 1: Determine a plane, referred to as a medial sagittal plane, that passes through a medial epicondyle of a knee joint and is perpendicular to a line (i.e., a TEA) connecting the medial epicondyle and a lateral epicondyle; and similarly, determine a lateral sagittal plane that passes through the lateral epicondyle of the knee joint.

Step 2: Calculate PC values of two intersections between a target rotation axis and a medial side and a lateral side of the medial sagittal plane/lateral sagittal plane by using the foregoing method to assess stability of the target rotation axis.

It needs to noted that although an MRI technique is used in the foregoing example to acquire a PC value to assess the stability of the rotation axis, the same technical effect can be achieved by replacing MRI with CT because CT can also acquire 3D images. In addition, a 2D image of X-ray radiography may be turned into 3D imaging data by additionally taking one MRI [“Posterolateral structures of the knee in posterior cruciate ligament deficiency”, Am J Sports Med, No. 37, pp. 534-541, 2009], and further the same technical effect can also be obtained. In addition, the stability of each point on bones can also be obtained by replacing the knee joint in this example with another joint.

3. Concepts of an i-MSA and an a-MSA and methods for localizing the i-MSA and the a-MSA in images.

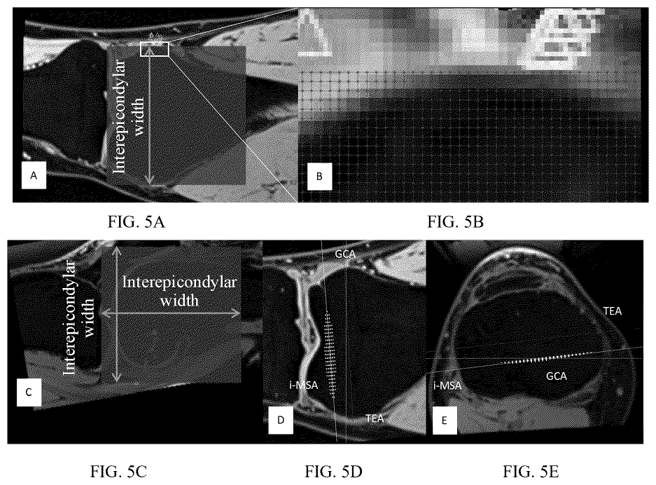

In MRI images of a knee joint, a 3D mesh that includes an entire distal end of a femur is established through 3D reconstruction software (for example, Mimics, where the software Mimics can convert the MRI images into a 3D model, and supports meshing), and a mesh spacing is 1 mm. All mesh intersections are used to form a candidate point set, and a PC value of each point is calculated. 0.2% of points with the smallest PC values are selected to form a most stable point group, and then a mathematical method is used (because a knee joint mainly performs flexion and extension, the most stable point group has a relatively simple distribution, and the points are approximately located on a same straight line, referring to FIG. 5D and FIG. 5E). Therefore, a simple mathematical method, for example, a least squares method, a principal component analysis method, and a gradient descent method, can be used. In this embodiment, a straight line that passes through the point group is fitted using the least squares method as the i-MSA (as shown in FIGS. 5A-5E). The i-MSA is the i-MSA. Theoretically, the surgical outcomes are optimal when a rotation axis of a knee arthroplasty (TKA or UKA) prosthesis is installed at the i-MSA. Due to time consumption (40 minutes to 50 minutes), the i-MSA measurement is not suitable for large-scale clinical application. Therefore, the concept of the a-MSA is proposed in this embodiment. In this embodiment, i-MSA measurement of 36 healthy subjects is first completed, and then a medial sagittal plane coordinate system and a lateral sagittal plane coordinate system are established using the method shown in FIG. 1A to FIG. 1D (in this embodiment, a unit length of the coordinate systems is an interepicondylar width (a distance between a medial epicondyle and a lateral epicondyle of a femur), or another unit, for example, millimeter, a body height, or a leg length may be used instead). All the methods used for the i-MSA (FIG. 1E and FIG. 1F) are turned into four parameters: M-PD, M-AP, L-PD, and L-AP. For the four parameters, average values of parameters corresponding to the i-MSAs of the 36 healthy subjects are taken respectively to obtain four corresponding parameters of the a-MSA:M-PD=22.2, M-AP=15.2, L-PD=20.3, and L-AP=17.7 (FIG. 1E and FIG. 1F). For a new subject, only one MRI needs to be taken to determine a position of an a-MSA through the medial sagittal plane coordinate system and the lateral sagittal plane coordinate system. A laborious measurement process for the i-MSA is avoided, so that a total measurement time is reduced to 4 minutes to 5 minutes.

A method for acquiring an i-MSA provided in this embodiment includes the following steps:

Step 1: Take MRI images of a joint of a subject at different angles using the foregoing method, and acquire values of a 6-DOF relative movement between bones at different bending angles.

Step 2: Perform 3D meshing with a preset mesh size on an individual target joint through a computer-aided tool (3D reconstruction software, for example, Mimics, where the software Mimics can convert the MRI images into a 3D model), randomly select one point in each mesh to form a candidate point set; and calculate PC values of all points in the candidate point set using the foregoing method according to the values of the relative movement obtained in step S1, and select the preset proportion of first points with the smallest PC values as the most stable point group, referring to FIGS. 5A-5E. When a unified mesh obtained by splicing all meshes is a standard body, the preset proportion may be 0.2%. The standard body here is the smallest cube that can accommodate an entire bone at a distal end of a bone on a side of the joint close to a human head; and when a volume of the unified mesh is different from that of the standard body, the preset proportion for selecting points is adjusted according to an inverse proportion of the volumes of the unified mesh and the standard body.

Step 3: Determine one straight line closest to all points in the most stable point group using a mathematical method, for example, a least squares method, as an i-MSA.

A method for acquiring an a-MSA provided in this embodiment includes the following steps:

Step 1: Acquire i-MSA of joints of a plurality of subjects according to the method for acquiring an individual MSA; then acquire a TEA of a joint of a corresponding subject through a computer-aided measurement tool, where specifically, the computer-aided measurement tool may be 3D modeling software, preferably, Mimics, Catia, or the like; and perform 3D reconstruction on the joint of the subject based on scan data of a CT device or an MRI device and by using the 3D modeling software (for example, Mimics, and Catia), and measure a TEA on a virtual model.

Step 2: Determine, through the computer-aided measurement tool, a 3D space coordinate system in which a joint is located: using a straight line in which a TEA is located as an X axis; and then drawing an inscribed circle of a diaphyseal medullary cavity of a femur/humerus at a transverse height of a standard length on a proximal side of the TEA, and drawing a perpendicular line from a center of circle to the X axis as a Z axis, where a direction of a Y axis is perpendicular to both the X axis and the Z axis, referring to FIGS. 1A-1F. Next, a medial sagittal plane and a lateral sagittal plane are determined: using a plane that is perpendicular to the X axis and passes through a medial epicondyle of the femur/humerus as a medial sagittal plane, and using a plane that is perpendicular to the X axis and passes through a lateral epicondyle of the femur/humerus as a lateral sagittal plane; on the medial sagittal plane, establishing a 2D coordinate system, referred to as the medial sagittal plane coordinate system, with the medial epicondyle of the femur/humerus as an origin and the Y axis and the Z axis as coordinate axis directions; and on the lateral sagittal plane, establishing a 2D coordinate system, referred to as the lateral sagittal plane coordinate system, with the lateral epicondyle of the femur/humerus as an origin and the Y axis and the Z axis as coordinate axis directions. A unit length of the coordinate systems is 1/200 of an interepicondylar width (i.e., a distance between a medial end and a lateral end of the TEA). Positions of intersections between an i-MSA and the medial sagittal plane and the lateral sagittal plane are described using the coordinate systems, and there are a total of four parameters: M-PD, M-AP, L-PD, and L-AP. Average values are taken for the four parameters respectively as corresponding parameters of an a-MSA, i.e., a population average relative position relationship between the individual MSA and the TEA, to acquire an a-MSA. A distance between the TEA in FIG. 1D and the center of circle of an inscribed circle in a medullary cavity of a femur may be any value of a multiple ranging from 0.44 times to 1 time of the interepicondylar width, provided that the value is the same for all the subjects. When the multiple is less than 0.44, the inscribed circle may still be located inside a knee joint/an elbow joint and fail to reach a diaphysis. When the multiple is greater than 1.0, the practical value is reduced because more diaphyses need to exposed upward during surgery for localization, leading to increased surgical trauma.

The stability of the i-MSAs and the a-MSAs of the 36 healthy subjects is shown in Table 2. It can be seen that the PC value of the i-MSA is significantly less than that of the a-MSA, the PC value of the a-MSA is significantly less than that of the TEA, and the PC values of a GCA and the a-MSA are not significantly different. It indicates that the i-MSA is more stable than the GCA, the GCA is more stable than the TEA, and the a-MSA and the GCA have close stability. In addition, because OA-induced bone defects affect the localization of the GCA, it is generally not possible to localize the GCA in patients undergoing knee arthroplasty. Therefore, the GCA does not have practical surgical value. In summary, both the i-MSA and the a-MSA have superior stability compared to conventional rotation axes, and the a-MSA is fast and convenient to measure and is readily suitable for large-scale application.

The stability of conventional 3D rotation axes (TEAs and GCAs) of the 36 subjects is shown in Table 2 below.

TABLE 2
PC values of two intersections between the a-MSA and the medial
sagittal plane/lateral sagittal plane and comparison results
of the PC values with those of the TEA, the GCA, and the i-MSA

| Intersections with the medial sagittal plane | | | |
|---|---|---|---|
| and lateral sagittal plane | | PC (mm) | P value* |
| a-MSA | Medial intersection | 5.9 ± 1.0 | |
| | Lateral intersection | 6.3 ± 1.2 | |
| TEA | Medial intersection | 10.6 ± 2.1 | 0.000 |
| | Lateral intersection | 11.5 ± 1.9 | 0.000 |
| GCA | Medial intersection | 6.3 ± 1.0 | 0.059 |
| | Lateral intersection | 6.9 ± 2.0 | 0.093 |
| i-MSA | Medial intersection | 3.8 ± 0.2 | 0.000 |
| | Lateral intersection | 4.1 ± 0.4 | 0.000 |

*P < 0.05 is a significant difference

It needs to noted that although an MRI technique is used in the foregoing example to acquire the i-MSA and the a-MSA, the same technical effect can be achieved by replacing MRI with CT because CT can also acquire 3D images. In addition, a 2D image of X-ray radiography may be turned into 3D imaging data by additionally taking one MRI [“Posterolateral structures of the knee in posterior cruciate ligament deficiency”, Am J Sports Med, No. 37, pp. 534-541, 2009], and further the same technical effect can also be obtained. In addition, the stability of each point on bones can also be obtained by replacing the knee joint in this example with another joint, to obtain the i-MSA. An elbow joint also has an anatomical structure (a line connecting a medial epicondyle and a lateral epicondyle of a humerus) similar to that of a TEA of a knee joint, and therefore the method for acquiring an a-MSA is also applicable to an elbow joint.

The above is only preferred embodiments of the present invention and is not intended to limit the scope of the patent of the present invention. Any equivalent structure transformation made using the content of the specification of the present invention and the accompanying drawings under the inventive concept of the present invention, or any direct/indirect application in other related technical fields, falls within the scope of protection of the patent of the present invention.

What is claimed is:

1. A method for measuring a relative movement between bones, comprising the following steps:
S11: imaging, by a magnetic resonance imaging (MRI) device, the bones before a movement for a first time to obtain a first image;
S12: imaging, by the MRI device, the bones after the movement for a second time to obtain a second image, and then rotating the second image to make an orientation of the imaging of the bones in the second image same as an orientation of the imaging of the bones in the first image, wherein a reference for determining whether the orientations are the same is to compare characteristic area frameworks (CAFs) to determine whether the CAFs are consistent, each of the CAFs is formed based on a plurality of characteristic areas in the bones, and the characteristic areas are subcortical vessels (SCVS) of the bones; and
S13: measuring, by the MRI device, a difference between angles of the second image before the rotation and after the rotation and a displacement distance between the second image after the rotation and the first image, and calculating a value of the relative movement between the bones based on the difference between the angles and the displacement distance.

2. The method according to claim 1, wherein in step 12, the characteristic areas are areas in which vessels lie immediately beneath bone cortices and traverse the bone cortices.

3. The method according to claim 1, wherein in step 12, the CAFs comprise at least two characteristic areas in different slices in an MRI image, and an interval between the at least two characteristic areas is not less than a preset quantity of MRI slices; and a size of each of the at least two characteristic areas does not exceed a preset size limit.

4. A method for acquiring an individual bone joint most stable axis (i-MSA), used for searching for a joint rotation axis in arthroplasty for humans, and comprising the following steps:
S1: acquiring values of a 6-degree-of-freedom (DOF) relative movement between bones of an individual target joint at a plurality of bending angles based on the method for measuring the relative movement between the bones according to claim 1 or a conventional method for measuring the relative movement between the bones;
S2: performing three-dimensional (3D) meshing with a preset mesh size on the bones of the individual target joint through a computer-aided tool, and randomly selecting a point in each mesh to form a candidate point set;
S3: quantitatively assessing a stability of each point in the candidate point set in step S2 using the values of the 6-DOF relative movement obtained in step S1, and selecting a preset quantity of most stable points to form a most stable point group; and
S4: finding a straight line closest to all points in the most stable point group using a least squares method or gradient descent method as an i-MSA of the individual target joint.

5. The method according to claim 4, wherein in step S3, a method for assessing the stability of the point is as follows:
using a point with stability to be assessed as a target point, and obtaining, according to the values of the 6-DOF relative movement between the bones of the individual target joint at the plurality of bending angles, a position coordinate of the target point at each of the plurality of bending angles; and
then calculating an average coordinate of the position coordinates, measuring a distance of the average coordinate from each of the position coordinates, and then calculating a mean square error or an arithmetic average value of the distances, wherein when the mean square error or the arithmetic average value is larger, a stability of the target point is poorer, and when the mean square error or the arithmetic average value is smaller, the stability of the target point is better.

6. The method according to claim 4, wherein in step S3, a method for acquiring the preset quantity of the points in the most stable point group is as follows:
when a unified mesh obtained by splicing all meshes in step S2 is a standard body, selecting a preset proportion of points with a best stability as the most stable point group, wherein the standard body is a smallest cube, the smallest cube is configured to accommodate an entire bone at a distal end of a bone on a side of a joint adjacent to a human head; and when a volume of the unified mesh is different from a volume of the standard body, adjusting, according to an inverse proportion of the volume of the unified mesh and the volume of the standard body, the preset proportion for selecting the point.

7. A method for acquiring a population average most stable axis (a-MSA) applicable to knee joints and elbow joints, comprising the following steps:
S1: acquiring transepicondylar axes (TEAs) of joints under test of a plurality of targets through a computer-aided measurement tool;
S2: acquiring individual most stable axes of all the joints under test in step S1 based on the method according to claim 4;
S3: determining, through the computer-aided measurement tool, a 3D space coordinate system in which the joint under test is located: using a straight line in which the TEA is located as an X axis; and then drawing an inscribed circle of a diaphyseal medullary cavity of a femur/humerus at a transverse height of a standard length on a proximal side of the TEA, and drawing a perpendicular line from a center of the inscribed circle to the X axis as a Z axis, wherein a direction of a Y axis is perpendicular to both the X axis and the Z axis;
S4: determining a medial sagittal plane coordinate system and a lateral sagittal plane coordinate system through the computer-aided measurement tool: using a plane that is perpendicular to the X axis and passes through a medial epicondyle of the femur/humerus as a medial sagittal plane, and using a plane that is perpendicular to the X axis and passes through a lateral epicondyle of the femur/humerus as a lateral sagittal plane; on the medial sagittal plane, establishing a two-dimensional (2D) coordinate system, referred to as the medial sagittal plane coordinate system, with the medial epicondyle of the femur/humerus as an origin and the Y axis and the Z axis as coordinate axis directions; and on the lateral sagittal plane, establishing a 2D coordinate system, referred to as the lateral sagittal plane coordinate system, with the lateral epicondyle of the femur/humerus as an origin and the Y axis and the Z axis as coordinate axis directions; and
S5: for a first intersection between the individual MSA of each joint under test and the medial sagittal plane, representing a first anterior-posterior position, i.e., a Y-axis coordinate, of the first intersection in the medial sagittal plane coordinate system using a parameter medial-anterior-posterior (M-AP); representing a first proximal-distal position, i.e., a Z-axis coordinate, of the first intersection using a parameter medial-proximal-distal (M-PD); representing a second anterior-posterior position, i.e., a position on the Y axis, of a second intersection between the individual MSA and the lateral sagittal plane using a parameter lateral-anterior-posterior (L-AP); representing a second proximal-distal position, i.e., a position on the Z axis, of the second intersection using a parameter lateral-proximal-distal (L-PD); and taking population average values of the four parameters, i.e., a population average relative position relationship between the individual MSA and the TEA, to acquire the a-MSA.

8. A method for acquiring a population average most stable axis (a-MSA) applicable to knee joints and elbow joints, comprising the following steps:
S1: acquiring TEAs of joints under test of a plurality of targets through a computer-aided measurement tool;
S2: acquiring individual most stable axes of all the joints under test in step S1 based on the method according to claim 5;
S3: determining, through the computer-aided measurement tool, a 3D space coordinate system in which the joint under test is located: using a straight line in which the TEA is located as an X axis; and then drawing an inscribed circle of a diaphyseal medullary cavity of a femur/humerus at a transverse height of a standard length on a proximal side of the TEA, and drawing a perpendicular line from a center of the inscribed circle to the X axis as a Z axis, wherein a direction of a Y axis is perpendicular to both the X axis and the Z axis;
S4: determining a medial sagittal plane coordinate system and a lateral sagittal plane coordinate system through the computer-aided measurement tool: using a plane that is perpendicular to the X axis and passes through a medial epicondyle of the femur/humerus as a medial sagittal plane, and using a plane that is perpendicular to the X axis and passes through a lateral epicondyle of the femur/humerus as a lateral sagittal plane; on the medial sagittal plane, establishing a 2D coordinate system, referred to as the medial sagittal plane coordinate system, with the medial epicondyle of the femur/humerus as an origin and the Y axis and the Z axis as coordinate axis directions; and on the lateral sagittal plane, establishing a 2D coordinate system, referred to as the lateral sagittal plane coordinate system, with the lateral epicondyle of the femur/humerus as an origin and the Y axis and the Z axis as coordinate axis directions; and
S5: for a first intersection between the individual MSA of each joint under test and the medial sagittal plane, representing a first anterior-posterior position, i.e., a Y-axis coordinate, of the first intersection in the medial sagittal plane coordinate system using a parameter medial-anterior-posterior (M-AP); representing a first proximal-distal position, i.e., a Z-axis coordinate, of the first intersection using a parameter medial-proximal-distal (M-PD); representing a second anterior-posterior position, i.e., a position on the Y axis, of a second intersection between the individual MSA and the lateral sagittal plane using a parameter lateral-anterior-posterior (L-AP); representing a second proximal-distal position, i.e., a position on the Z axis, of the second intersection using a parameter lateral-proximal-distal (L-PD); and taking population average values of the four parameters, i.e., a population average relative position relationship between the individual MSA and the TEA, to acquire the a-MSA.

9. The method according to claim 7, wherein in step S3, the standard length is a distance between a medial epicondyle and a lateral epicondyle of the TEA×a, wherein a is a coefficient and has a value ranging from 0.44 to 1.0.

10. The method according to claim 8, wherein in step S3, the standard length is a distance between a medial epicondyle and a lateral epicondyle of the TEA×a, wherein a is a coefficient and has a value ranging from 0.44 to 1.0.
